(19) United States
(12) Patent Application Publication
(10) Pub. No.: US 2012/0150206 A1
(43) Pub. Date: Jun. 14, 2012

(54) REINFORCING MEDICAL DEVICE

(75) Inventors: Michel Barikosky, Paris (FR); Christian Girardiere, Paris (FR)

(73) Assignee: LES LABORATOIRES BROTHIER, Nanterre (FR)

(21) Appl. No.: 13/315,087

(22) Filed: Dec. 8, 2011

(30) Foreign Application Priority Data
Dec. 8, 2010 (FR) 1060273

(51) Int. Cl. A61B 17/11

(52) U.S. Cl. 606/153

(57) ABSTRACT

Such reinforcing medical device for a sutured tissue area, comprising a stapling line and a stapling circle, the device having a ring shape, so as to cover and reinforce the area along the stapling circle, wherein the ring comprises covering and reinforcing side means (4,6) so as to cover and reinforce portions of the stapling line extending beyond the stapling circle.

The present invention relates to a medical device for reinforcing sutured tissue areas.

The invention finds particularly application in the field of mechanical anastomoses, aspecially in visceral surgery.

Anastomosis is a manual or mechanical surgery act consisting in restoring connection between two anatomic ducts. Such act is highly used in visceral or digestive surgery.

The digestive anastomoses refer as to the operation consisting, after a resection (cutting) of a part of the digestive tract, in restoring continuity by assembling both remaining pieces 101, 102 of the digestive tract. This operation type can be performed for instance in the case of a colorectal cancer, but also of inflammatory pathologies like Crohn's disease, ulcerative colitis, diverticulitis, etc.

Further to this type of operation, leaks 103 can occur at the anastomosis level and lead to serious complications which will delay patient curing at best or cause infections 104 at worst (FIG. 6).

Anastomoses can be performed either manually with sutures or mechanically with staplers.

The use these last years of mechanical techniques implementing linear and circular stapling clips has simultaneously made surgeon's gesture and patient security easier by reducing in particular the appearance of fistulas. However, the risk of complications like leaks or a stenosis and the mortality rate remain significant. In order to reduce such complications and such mortality rate, new strategies have been developed with the use of stapling reinforcing devices so as to decrease bleedings and reduce tensions on the adjacent tissues. Some of such reinforcing devices are absorbable, but others not. Examples of reinforcing devices may include PTFE (PolyTetraFluoroEthylene), bovine pericardium, cellulose-based resorbable polymer or porcine intestinal tissue devices.

However, such devices have numerous disadvantages, including a high price, an uncertain viral innocuousness as well as risks due to prions.

The present invention aims at improving the situation proposing a reinforcing device for reducing the risks related to the use of stapling clips upon mechanical anastomoses.

To this end, a first aspect of the present invention relates to a reinforcing medical device for a sutured tissue area, comprising a stapling line and a stapling circle, the device having a ring shape, to cover and reinforce the area along the stapling circle, wherein the ring comprises covering and reinforcing side means so as to cover and reinforce portions of the stapling line extending beyond the stapling circle.

Remarkably, thanks to the covering and reinforcing protecting side means thereof, the reinforcing device according to the first aspect of the present invention is particularly adapted for mechanical anastomoses using a double linear and circular stapling. The annular shape of the device allows the linear stapling area to be covered, and the side means allow portions of the linear stapling area outside the circular area to be covered and reinforced.

In the state of art, reinforcing devices having a ring shape and provided with adhesive strips are known. Such adhesive strips are exclusively provided for fastening the reinforcing device to a mechanical circular stapling clip. Once the reinforcing device is positioned on the sutured area, the strips are removed from the reinforcing device by pivoting the stapler in the anastomosis being immobilized, then by removing the clip. Thus, such devices have nothing to do with the device of the present application.

According to a preferred embodiment, the covering and protecting side means are covering and reinforcing wings.

Furthermore, it is known that the mechanical anastomoses lead to a risk of postoperative stenosis, i.e. a narrowing, being higher than the manual anastomoses. Such risk of stenosis also depends on the anatomic areas to be stapled, the patient's medical history and the proper anastomosis being performed, more particularly on the more or less significant tension of the anastomosis. The stenosis treatment usually consists in one or more candle or balloon mechanical dilatations until the stenosis gives up.

The use of a reinforcing device makes unfortunately such dilatations more difficult to perform due to the rigidifying of the reinforced sutured area.

The present invention aims at improving the situation providing a reinforcing device without such disadvantage.

To this end, a second aspect of the present invention relates to a reinforcing medical device for a sutured tissue area, comprising a stapling circle, the device having a ring shape, so as to cover and reinforce the area along the stapling circle, wherein the ring comprises at least one notch located in a peripheral part of the ring and arranged to be situated perpendicularly to the stapling circle.

Thanks to said notch, a discontinuity is thus formed in the reinforcement of the sutured area, making such area more flexible. Such flexibility allows remarkably the treatment by dilatation to be made easier in case of a stenosis.

Preferably, the notch has a shape of a “U”, a “V” or a lying “C”, which, in the periphery of the reinforcing device, spreads its edges.

Advantageously, such a notch can be provided on the reinforcing device according to the first aspect of the present invention. This enables to get an efficient reinforcing device adapted for the anastomoses using a double linear and circular stapling on a tissue area having a high risk of stenosis.

The reinforcing devices according to the first and second aspects of the invention allow both to solve the same problem of reducing the risk of leaks in a sutured area upon a mechanical anastomosis. They result from a same general inventive concept.

More particularly, the reinforcing device provided with side means according to the first aspect of the invention allows the risk of leaks to be reduced within an anastomosis with a double linear and circular stapling.

On its side, the reinforcing device provided with notches according to the second aspect of the invention brings a quite useful flexibility for the practitioner, especially to make easier a treatment by dilatation upon a postoperative stenosis, either for an anastomosis by a single circular stapling or for an anastomosis by a double linear and circular stapling.

The present invention thus provides a reinforcing device minimizing the risks of leaks upon a mechanical anastomosis. The embodiment alternatives of the first and second aspects of the invention will be chosen by the surgeon depending on the tissue area to be stapled (with a more or less high risk of stenosis) and the technique for performing the anastomosis (single or double stapling).

Advantageously, the reinforcing device is made from a calcium alginate textile.

This allows the improvement of healing in the re-connected tissue areas by reinforcing in a natural way, after a resorption of the device, the anastomosis and thereby avoiding leaks and fistulas appearing at the level of the whole of sutures.

According to another characteristic, the device is conforming while having a shape memory.

Such properties can be achieved by using a calcium alginate textile coated with a biocompatible and biodegradable polymer, for example propylene glycol alginate (PGA).

Such properties allow the device to follow the contours of the tissue areas so as to provide an optimal contact and guarantee consequently a good sealing and a good migration for the haemostatic and healing agents.

Moreover, those characteristics in terms of shaping and shape memory allow using the device in coelioscopy.

Embodiments of the invention will now be further described, but with no limitation, referring to the accompanying drawings, wherein:

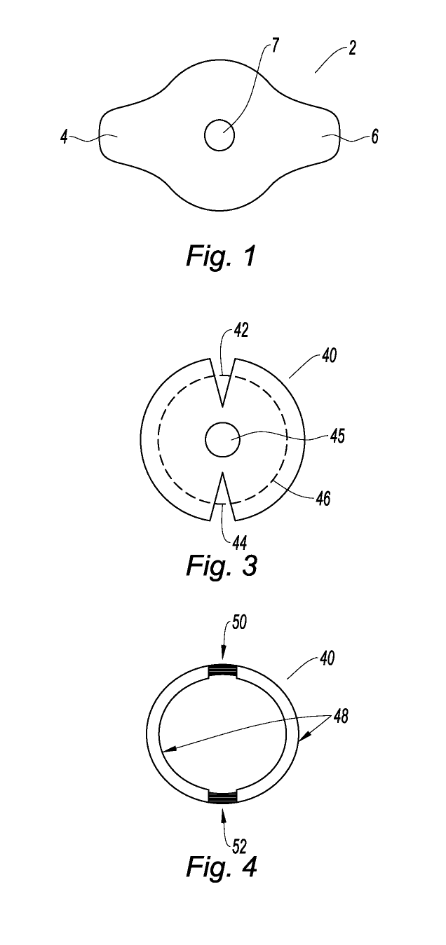

FIG. 1 shows a reinforcing device according to the first aspect of the invention;

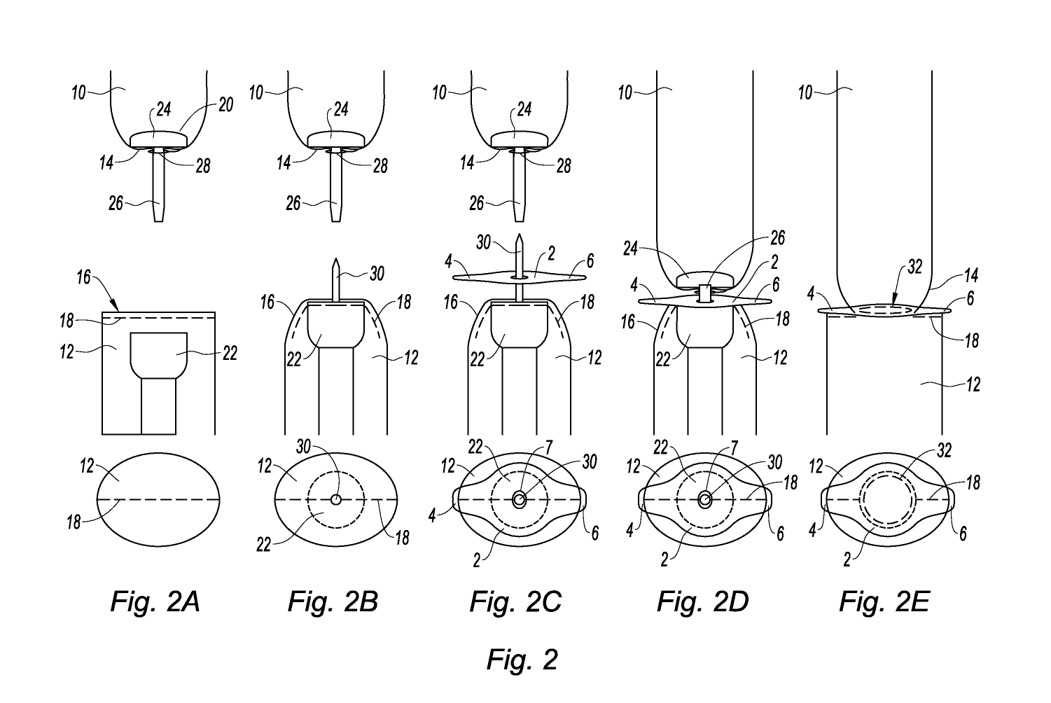

FIG. 2 shows the different steps (in side and top views) of an anastomosis with a double stapling using the reinforcing device of FIG. 1;

FIG. 3 shows a reinforcing device according to the second aspect of the invention;

FIG. 4 shows the state of the reinforcing device of FIG. 3 after a circular stapling;

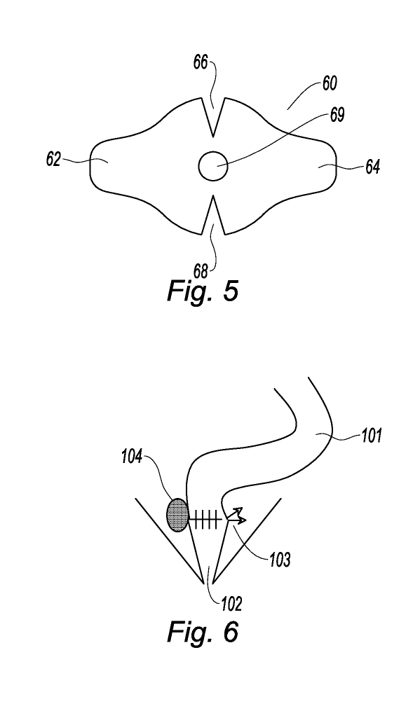

FIG. 5 shows a reinforcing device combining the characteristics of the device according to FIG. 1 and the device according to FIG. 3; and

FIG. 6 shows the problem of the anastomotic leaks.

FIG. 1 shows a reinforcing device 2 having a plane ring shape comprising side wings 4, 6 and a circular central hole 7.

The bioresorbable reinforcing device 2 is made from a calcium alginate textile, preferably coated on one of the two sides thereof with a biocompatible and biodegradable polymer solution such as for instance propylene glycol alginate (PGA).

The haemostatic and healing activity of calcium alginates is in fact recognized in the field of wound healing. The calcium rate on the one hand and the releasing speed thereof on the other hand are key parameters for controlling haemostasis and healing.

An alginate of a guluronic type is preferred to an alginate being rich in mannuronic motifs, since the latter releases rapidly calcium ions and does not allow to control the biological steps leading to the tissue healing.

According to a preferred embodiment, the textile of the reinforcing device 2 is a knitting obtained from a calcium alginate yarn with a size between 100 and 500 dTex (decitex). The molar ratio between the two basic monomers, the mannuronic acid M and the guluronic acid G, is selected so that M/G is lower than 1. This creates a roughened surface having a controlled porosity, preferably higher than 75 μm. Such a porosity allows to guarantee a strong fibroblastic reaction leading to the tissue integration of the material and thus to the adherence of the reinforcing device 2 with the tissue area. This reaction is due to the penetration of macrophages, fibroblasts and collagen fibers. On the contrary, a surface with a porosity being lower to 10 μm generates a low adherence. Preferably, the knitting stitch is a blocked jersey of “locked stitch” type with a gauge 32 having an inter-stitch diameter higher than 75 μm.

FIGS. 2A to 2E of FIG. 2 show the different steps of an anastomosis with double stapling, for example, a colorectal one using the reinforcing device 2.

FIG. 2A shows a colic part 10 and a rectal part 12 of a tissue area located on either side of a “sick” digestive part, for example having a tumour being resected in a colic end 14 and a rectal end 16. The colic part 10 is located on the colon side and the rectal part 12 is located on the rectum side. The resection at the colic end 14 is performed conventionally with a cold knife whereas the resection on the rectal end 16 is performed by means of a cutting linear stapler, not shown, so that a stapling line 18 is formed.

Further to the resection of the “sick” part, a circular stapling is performed by means of a cutting circular stapler comprising an anvil 20 and a clip body 22. The anvil 20 comprises a head 24, being preferably flexible, and a stem 26.

For this, the anvil 20 is inserted in the colic part 10 from the colic end 14. It is fastened at the level of such colic end by a yarn 28 clamping the colic end 14 around the stem 26.

Then, the clip body 22 is inserted, for example thru the rectum, in the rectal part 12.

A top view of the rectal part 12 is illustrated below of FIG. 2A, in which the stapling line 18 is well distinguished.

The next step, being illustrated in FIG. 2B, consists in the rectal end 16 being perforated by projecting a trocar 30 out of the clip body 22. It is the tip of the trocar that perforates the rectal end 16. The result of such perforation is illustrated on the top view of the rectal part 12 at the bottom of FIG. 2B.

After the rectal end 16 being perforated, the reinforcing device 2 is inserted on the trocar 30 in the way shown on FIG. 2C. In order to provide the reinforcing device 2 being maintained on the trocar 30, the diameter of the hole 7 is selected to be substantially equal to, preferably less than the diameter of the trocar 30.

A top view of the rectal part 12 further to such insertion of the reinforcing device is represented at the bottom of FIG. 2C.

FIG. 2D shows the next step consisting in drawing together the stem 26 of the anvil 20 and the trocar 30 so as to perform a circular stapling.

After the stem 26 and the trocar 30 being clicked on together, and then a circular stapling, a stapling circle designated by numeral 32 on FIG. 2E is then formed. The part of the reinforcing device 2, the circular part of the colic end 14 and the circular part on the rectal end 16 bounded by the stapling circle 32 are simultaneously cut.

The last operation performed by the surgeon consists then in removing the clip body 22 and the anvil 20. The removal of the anvil 20 is made easier thanks to the selection of the flexible head 24.

The final result of this double linear and circular stapling is illustrated on the top view of the rectal part 12 at the bottom of FIG. 2E.

Remarkably, the sutured tissue area is wholly covered by the reinforcing device 2, the side wings 4 allowing a perfect covering of the remaining portions of the stapling line 18 after cutting the part of the reinforcing device 2 bounded by the stapling circle 32. Thus, the risk of postoperative leaks is minimized.

FIG. 3 illustrates a reinforcing device 40 according a second aspect of the invention. The device 40 has the shape of a plane ring, comprising two peripheral notches 42, 44 and a central circular hole 45.

Advantageously, the reinforcing device 40 is made with the same calcium alginate textile as the reinforcing device 2 and possesses consequently the same properties in terms of bioresorbability, protection and healing of a tissue area as the reinforcing device 2.

The reinforcing devise 40 of FIG. 3 is adapted to be used upon a mechanical anastomosis by a single circular stapling, for example a duodeno-jejunal anastomosis, a gastroduodenal anastomosis and an esogastric anastomosis, a colo-colic anastomosis etc. Such anastomosis is performed on the same way as the anastomosis illustrated on FIGS. 2A to 2E with the exception of the linear stapling step.

Further to such circular stapling creating a sutured tissue area comprising a stapling circle 46, the reinforcing device 40 has the shape shown on FIG. 4. This device covers at the level of the peripheral areas 48 a big portion of the stapling circle 46 resulting from the anastomosis, thereby reducing the risk of any postoperative leaks.

Remarkably, thanks to the notches 42 and 44 arranged perpendicularly to the stapling circle 46, two narrow discontinuity areas 50 and 52 appear, at the level of which there is no covering of the tissue area. Such covering discontinuity areas, being well more flexible than the peripheral areas 48 being rigidified due to the reinforcement of the devise 40 allow a subsequent dilatation treatment to be easier in case of a postoperative stenosis.

Moreover, the presence of such discontinuity areas makes easier the removal of the anvil after the circular stapling, which makes the use of the reinforcing device 40 optimum even with an anvil without any flexible head.

FIG. 5 describes an embodiment alternative of the reinforcing device according to the invention combining the characteristics of the reinforcing devices 2 and 40.

Such a reinforcing device 60 has a plane ring shape. It comprises two side wings 62, 64, two peripheral notches 66, 68 arranged to be located perpendicularly to the stapling circle and a central circular hole 69.

It is particularly adapted for the anastomosis by double linear and circular stapling of tissue areas with a high risk of stenosis.

Obviously, other embodiments can be still envisaged.

Thus, a number of notches being different from two can be provided, for example one single notch or still a number of notches higher than two.

Shaping and shape memory characteristics of the reinforcing device of the invention have been mentioned above. Such characteristics are quite interesting. In fact, the reinforcing device must be able to be used in a coelioscopy, which needs the passage of the device thru the trocar. The device will thus be folded on itself. After passing thru the trocar, it is essential that the device finds again its initial shape for stapling with the anvil of the stapler.

1.-7. (canceled)

8. A reinforcing medical device for a sutured tissue area, comprising a stapling line and a stapling circle, the device having a ring shape, so as to cover and reinforce the area along the stapling circle, wherein the ring comprises recovering and reinforcing side means so as to cover and reinforce portions of the stapling line extending beyond the stapling circle.

9. A device according to claim 8, wherein the side means consist in covering and reinforcing wings.

10. A device according to claim 8, wherein the ring comprises at least one notch located in a peripheral part of the ring and arranged to be situated perpendicularly to the stapling circle.

11. A device according to claim 10, comprising two notches.

12. A device according to claim 9, wherein the ring comprises at least one notch located in a peripheral part of the ring and arranged to be situated perpendicularly to the stapling circle.

13. A device according to claim 12, comprising two notches.

14. A device according to claim 8, the device being made of calcium alginate textile.

15. A device according to claim 8, the device being conforming and showing a shape memory.

16. A reinforcing medical device for a sutured tissue area, comprising a stapling circle, the device having a ring shape, so as to cover and reinforce the area along the stapling circle, wherein the ring comprises at least one notch located in a peripheral part of the ring and arranged to be situated perpendicularly to the stapling circle and to form a discontinuity in the reinforcement of the sutured area, making such area more flexible.

17. A device according to claim 16, comprising two notches.

18. A device according to claim 16, the device being made of calcium alginate textile.

19. A device according to claim 16, the device being conforming and showing a shape memory.
